SEQUENCE LISTING (24 sequences)

SEQ ID NO: 1 - DNA, 1197 bp, Yarrowia lipolytica
atgagcgctctacttcgatcgtccctgcgttttaaacacatgtccgccgtcaaccgtctcacacaacagcttcgactgctgaccgcctccgcgcctctcagcgcagccaacaccgccggcaaggctcctttcaaggtcgccgttgttggttctggtaactggggaaccaccgtcgccaagattgtcgccgagaactgcactgctcaccccgagctctttgagcccgaggttcgagtctgggttcgagaagagaaggtcaacggcaagaacctgaccgacattttcaacgctgagcacgagaacgtgcgatacctccctaaaatcaaacttcctcacaacctgatcgccgagccggatctgctcaaggccgtcgagggtgccaacatcatcgtcttcaacctgccccatcagttcctggctggtgtctgcaagcagctcaagggccacgtcaaccccaaggctagagccatctcctgcctcaagggtctagatgtcaccccccagggtgtttacctgctctccgacgttatcgagaacgagaccggtctccactgcggtgttctgtccggggctaacctcgccaccgagatcgctctggagaagtactccgagactaccgttgcttacaaccgacccaaggacttctttggcgagggtgatgtgaccaacgatgtgctcaaggctctgttccaccgaccctacttccatgtgcgatgcgttcaggacgtcgccggtgtctccatcggaggtgcccttaagaacgttgttgccctttgcgccggtttcgtcgagggcaagaactggggagacaacgccaaggccgcaattatgcgacgaggcatgcttgagatgatcaacttctccaagcgattcttccccgaaactgatattaacactcttacagtcgagtctgccggtgtggccgatctcatcacctcgtgcgctggaggccgaaacttcaaggtcggccgagcattcggaaaggagagcggctccggcaagaccatccaggacgtggagaaggagcttctcaacggccagtccgcccagggcgtcatcacatgcaacgaggtccacgagctgctcaagaacaagaacatgcagaaggacttccctctgttcgagtccacctggggcattatccacggtgagctcaagattgatgatctccccgagattctttaccacgccaactag

SEQ ID NO: 2 - protein, 398 aa, Yarrowia lipolytica
MSALLRSSLRFKHMSAVNRLTQQLRLLTASAPLSAANTAGKAPFKVAVVGSGNWGTTVAKIVAENCTAHPELFEPEVRVWVREEKVNGKNLTDIFNAEHENVRYLPKIKLPHNLIAEPDLLKAVEGANIIVFNLPHQFLAGVCKQLKGHVNPKARAISCLKGLDVTPQGVYLLSDVIENETGLHCGVLSGANLATEIALEKYSETTVAYNRPKDFFGEGDVTNDVLKALFHRPYFHVRCVQDVAGVSIGGALKNVVALCAGFVEGKNWGDNAKAAIMRRGMLEMINFSKRFFPETDINTLTVESAGVADLITSCAGGRNFKVGRAFGKESGSGKTIQDVEKELLNGQSAQGVITCNEVHELLKNKNMQKDFPLFESTWGIIHGELKIDDLPEILYHAN

SEQ ID NO: 3 - DNA, 37 bp, Artificial Sequence
  Oligonucleotide
gcggatcccacaatgagcgctctacttcgatcgtccc

SEQ ID NO: 4 - DNA, 36 bp, Artificial Sequence
  Oligonucleotide
gcgcctaggctagttggcgtggtaaagaatctcggg

SEQ ID NO: 5 - DNA, 18 bp, Artificial Sequence
  Oligonucleotide
tccaggccgtcctctccc

SEQ ID NO: 6 - DNA, 21 bp, Artificial Sequence
  Oligonucleotide
ggccagccatatcgagtcgca

SEQ ID NO: 7 - DNA, 19 bp, Artificial Sequence
  Oligonucleotide
tcctcaagcgacacgtcta

SEQ ID NO: 8 - DNA, 18 bp, Artificial Sequence
  Oligonucleotide
cagcaacagcaggtatcc

SEQ ID NO: 9 - DNA, 22 bp, Artificial Sequence
  Oligonucleotide
ttctcatcttccagtacgccta

SEQ ID NO: 10 - DNA, 21 bp, Artificial Sequence
  Oligonucleotide
ggcaataagattgagaccgtt

SEQ ID NO: 11 - DNA, 18 bp, Artificial Sequence
  Oligonucleotide
tgtaccgattccagcagt

SEQ ID NO: 12 - DNA, 19 bp, Artificial Sequence
  Oligonucleotide
ggtgtgggagataaggcaa

SEQ ID NO: 13 - DNA, 19 bp, Artificial Sequence
  Oligonucleotide
cgagactaccgttgcttac

SEQ ID NO: 14 - DNA, 19 bp, Artificial Sequence
  Oligonucleotide
caacaacgttcttaagggc

SEQ ID NO: 15 - DNA, 21 bp, Artificial Sequence
  Oligonucleotide
tgtgtggaacctcgactacaa

SEQ ID NO: 16 - DNA, 19 bp, Artificial Sequence
  Oligonucleotide
catccagaagtagggaagc

SEQ ID NO: 17 - DNA, 19 bp, Artificial Sequence
  Oligonucleotide
ctccgccgacttctttatg

SEQ ID NO: 18 - DNA, 18 bp, Artificial Sequence
  Oligonucleotide
gaagtatccgtctcggtg

SEQ ID NO: 19 - DNA, 18 bp, Artificial Sequence
  Oligonucleotide
gagattgtgtccgacact

SEQ ID NO: 20 - DNA, 20 bp, Artificial Sequence
  Oligonucleotide
tttgtcgaatacggatcggt

SEQ ID NO: 21 - DNA, 20 bp, Artificial Sequence
  Oligonucleotide
caagattgtctctccctacg

SEQ ID NO: 22 - DNA, 19 bp, Artificial Sequence
  Oligonucleotide
gtactcggatcaggttcac

SEQ ID NO: 23 - DNA, 19 bp, Artificial Sequence
  Oligonucleotide
gttcgacaaggagcctatt

SEQ ID NO: 24 - DNA, 21 bp, Artificial Sequence
  Oligonucleotide
ggtcagatgcggatgataaag

(19) United States
(12) Patent Application Publication
(10) Pub. No.: US 2013/0149754 A1
(43) Pub. Date: Jun. 13, 2013

(54) Optimization of Lipid Synthesis and Accretion

(71) Applicants: Thierry Dulermo, Saint Germain En Laye (FR); Jean-Marc Nicaud, Trappes (FR)

(72) Inventors: Thierry Dulermo, Saint Germain En Laye (FR); Jean-Marc Nicaud, Trappes (FR)

(21) Appl. No.: 13/806,332

(22) PCT Filed: Jul. 1, 2011
(86) PCT No.: PCT/EP2011/061106

§ 371 Date: Feb. 7, 2013

(30) Foreign Application Priority Data
Jul. 1, 2010 (FR) 1055331

(51) Int. Cl. C12P 7/64

(52) U.S. Cl. CPC C12P 7/6409
USPC 435/134; 435/471; 435/254.2; 435/254.22

(57) ABSTRACT

The present invention relates to a novel mutant strain of yeast, particularly a strain of Yarrowia lipolytica, which is capable of accumulating a large quantity of lipids. Said strain is deficient in the beta-oxidation of fatty acids and overexpresses the GPD1 gene. Preferably, the mutant strains, which do not express the GUT2 gene and which are deficient for the beta-oxidation of fatty acids, do not express the POX2 to POX5 genes, very preferably the POX2 to 6 genes. The invention also relates to a method for obtaining the strains of yeast according to the invention and to the use of said strains in the production of lipids.

Several technologies such as large-scale fermentation are used for the industrial production of oil from microorganisms by using fatty substances or glycerol as a substrate. Within the framework of these projects, the microorganisms are used as a cell factory by redirecting the metabolism thereof to the production of compounds of industrial or dietary interest, such as waxy esters, isoprenoids, polyhydroxyalkanoates and hydroxylated fatty acids. The majority of these target the production of reserve lipids with a specific structure and/or composition. These include essential polyunsaturated fatty acid-enriched oils, which can potentially be used as a food supplement, lipids having compositional similarities with cocoa butter and non-specific oils intended for use in synthesizing biofuels.

Consequently, a growing interest is being observed in improving the composition and oil content of microorganisms, particularly yeasts.

Oleaginous microorganisms are organisms capable of accumulating lipid reserves possibly exceeding 20% of the dry matter of the cell. Among these microorganisms the yeast Yarrowia lipolytica is one of the most studied and used, by reason of the capacity thereof to accumulate cell lipids of up to 40% of its dry weight.

Yeasts, in particular Yarrowia lipolytica, are capable of effectively using hydrophobic substrates, e.g., alkanes, fatty acids and oils as a single source of carbon (Fickers et al., FEMS Yeast Research, 5(6-7): 527-543, 2005). Ingested aliphatic chains can be used for energy production or accumulated in unchanged or modified forms.

Storage molecules such as triglycerides (TG) and/or sterol esters (sterylesters; SE), which are incapable of being integrated into the phospholipids bilayers, group together in order to form the hydrophobic nucleus of lipid bodies (LB).

Said lipid bodies were for a long time only considered to be storage for neutral lipids capable of being mobilized during a period of deprivation. However, the image of said lipid bodies as a simple storage compartment had to be revised since numerous proteins of said lipid bodies were identified as enzymes involved in lipid metabolism, in particular in the synthesis and/or degradation of triglycerides.

In yeasts, triglyceride synthesis follows the Kennedy pathway. The free fatty acids are activated for the coenzyme A (CoA) and used for the acylation of glycerol, which is pivotal to the synthesis of the triglycerides.

In the first step of assembling triglycerides, glycerol-3-phosphate (G-3-P) is acylated via the specific acyltransferase of the glycerol-3-phosphate (glycerol-3-phosphate acyltransferase or SCrT) in order to yield lysophosphatidic acid, which is then acylated via the specific acyltrasferase of the lysophosphatidic acid (phosphatidic acid acyltranferase or SLC1) in order to yield phosphatidic acid (PA). The latter is then dephosphorylated via a specific phosphohydrolase of the phosphatidic acid (phosphatidic acid phosphohydrolase (PAP)) in order to release diacylglycerol (DAG).

In the final step, the diacylglycerol is acylated either by diacylglycerol acyltransferase or by phospholipid diacylglycerol acyltransferase, in order to produce triglycerides.

Table 1 describes the genes involved in the metabolism of the fatty acids in yeasts, particularly in Yarrowia lipolytica, (YL). The sequences can be accessed by their names or accession numbers at the address: http://cbi.labri.fr/Genolevures/index.php#.

In yeasts, degradation of the fatty acids occurs via beta-oxidation, a process consisting of several steps requiring four different enzyme activities. In yeasts, the enzymes are substantially localized in peroxisomes, contrary to the case of mammals where they are localized in mitochondria and peroxisomes.

Mobilization of the accumulated lipids occurs in three separate phases:
(i) during the exponential phase, the stored lipid compounds are used for synthesizing the membrane lipids, so as to support cell growth and division,
(ii) during the stationary phase, when the nutrients are depleted, the free fatty acids are released rather slowly from the triglycerides and subjected to peroxisomal β-oxidation;
(iii) at the end of the deficiency phase, e.g., when the cells exit the stationary phase and re-enter the vegetative growth phase with carbon supplementation, the lipid deposits are very quickly degraded into free fatty acids.
Gylcerol-3-phosphate (G3P) is a critical player in the metabolism of the triglycerides.

There are two synthetic pathways for glycerol-3-phosphate.
In the first pathway, the glycerol-3-phosphate is derived from glycerol by means of the glycerol kinase encoded by the gene GUT1.
In the second pathway, the glycerol-3-phosphate is directly synthesized from dihydroxyacetone phosphate (DHAP). This reaction is catalyzed by the glycerol-3-phosphate dehydrogenase (encoded by the gene GPD1). This last reaction is reversible and the formation of DHAP from glycerol-3-phosphate is catalyzed by a second isoform of the glycerol-3-phosphate dehydrogenase, the gene of which is named GUT2.

The present inventors have previously shown that the inactivation of the gene GUT2 results in an increased accumulation of lipids in yeasts, particularly in Yarrowia lipolytica (WO 2010/004141; Beopoulous et al., Appl Environ Microbiol., 74(24); 7779-7789, 2008). The gene GUT2 encodes the isoform Gut2p of the glycerol-3-phosphate dehydrogenase, which catalyzes the oxidation reaction of the glycerol-3-phosphate into DHAP.

Surprisingly and unexpectedly, the overexpression of the glycerol-3-phosphate dehydrogenase encoded by the gene GDP1 (SEQ ID NO.: 1; SEQ ID NO.: 2), which is the enzyme catalyzing the synthesis reaction of glycerol-3-phosphate from DHAP, has no effect on the accumulation of lipids in a strain that is otherwise wild. In the same way, the overexpression of GPD1 in an inactivated mutant for the gene GUT2 has no effect on the accumulation of lipids in yeasts.

On the other hand, the inventors have shown that it is possible to obtain an accumulation of lipids by overexpressing the gene GPD1 in yeasts in which the beta-oxidation of the fatty acids is deficient.

This accumulation is much more significant than anything that has been previously described in the prior art. In particular, even though the strains in which GUT2 is inactivated accumulate at most 50% of lipids when they are cultivated in the presence of oleic acid (WO 2010/004141; Beopoulous et al., Appl Environ Microbiol., 74(24); 7779-7789, 2008), the strains of the invention, which are deficient in beta-oxidation, and which overexpress the gene GPD1, accumulate more than 60% of lipids under the same conditions.

Furthermore, the strains having a beta-oxidation deficiency and overexpressing the gene GPD1 have the particular feature of easily lysing, which is not the case with strains of the prior art. This property provides a sure benefit from an industrial viewpoint, since it facilitates cell lysis in order to recover the lipids produced.

The present invention therefore relates to a strain of yeast in which the beta-oxidation of the fatty acids is deficient and which overexpresses the gene GPD1, said mutant strain being capable of accumulating lipids.

Within the meaning of the present invention, the term “yeast” is understood to mean yeast strains in general, i.e., this term includes, among others, Saccharomyces cerevisiae, Saccharomyces sp., Hansenula polymorpha, Schizzosaccheromyces pombo, Yarrowia lipolytica, Pichia pastoris, Pichia finlandica, Pichia trehalophila, Pichia koclamae, Pichia membranaefaciens, Pichia minuta (Ogataea minuta, Pichia linderneri), Pichia opuntiae, Pichia thermotolerans, Pichia salictaria, Pichia guercuum, Pichia pijperi, Pichia stiptis, Pichia methanolica, Pichia sp., Kluyveromyces sp., Kluyveromyces lactis, Candida albicans.

Within the meaning of the invention, the preferred yeasts are the oleaginous yeasts (Ratledge, in: Ratlege C, Wilkinson S G editors, Microbial lipids, Vol. 2. London: Academic press 1988). Although the overexpression of the gene GPD1 in S. cerevisiae, in any genetic base, results substantially in an accumulation of glycerol, in the oleaginous yeasts deficient with regard to the beta-oxidation of the fatty acids, it results in an accumulation of lipids.

The present invention therefore preferentially relates to an oleaginous strain of yeast in which the beta-oxidation of the fatty acids is deficient and which overexpresses the gene GPD1, said mutant strain being capable of accumulating lipids.

The most widely known oleaginous yeasts include the genera Candida, Cryptoccocus, Rhodotorula, Rhizopus, Trichosporon, Lypomyces and Yarrowia. The particularly preferred yeasts, within the meaning of the invention, include Yarrowia lipolytica, Rhodotura glutinis and Rhodosporidium torulides. A preferred yeast within the meaning of the present invention is Yarrowia lipolytica.

Beta-oxidation is the degradation pathway for fatty acids. It involves 4 consecutive reactions during which an acyl-CoA oxidase is involved, of which the 6 isoforms are encoded by the 6 POX genes, a multifunctional enzyme is encoded by the MFE1 gene and a 3-oxoacyl-CoA thiolase is encoded by the POT1 gene. As indicated above, beta-oxidation in yeasts occurs exclusively in the peroxisome, a cytoplasmic organelle the biogenesis of which is controlled by PEX genes (see Table 2). When the peroxisome is not correctly assembled, or when it is not functional, the fatty acids are not properly degraded (WO 2006/064131; Thevenieau et al., Fungal Genet Biol., 44(6): 531-42, 2007).

Generally speaking, the mutations affecting beta-oxidation according to the invention are loss-of-function mutations which result in a very strong decrease or even a total absence of beta-oxidation. The loss-of-function mutations according to the invention can correspond to point mutations, insertions, total or partial deletions, gene replacements or any other molecular cause that results in a substantial decrease in beta-oxidation.

Within the meaning of the invention, the yeast strains in which the beta-oxidation of fatty acids is deficient include any strains having at least one loss-of-function mutation in at least one gene encoding an enzyme directly involved in beta-oxidation, but also all of the strains that have at least one loss-of-function mutation that only affects beta-oxidation indirectly, in particular through biogenesis or the function of the peroxisomes. It is well understood that the strains according to the invention also include all of the strains having combinations of the above-described mutations. For example, also within the scope of the present invention are the strains which have at least one loss-of-function mutation directly affecting beta-oxidation and at least one loss-of-function mutation affecting beta-oxidation only indirectly.

According to a preferred aspect of the invention, the strains deficient in the beta-oxidation of fatty acids include any strain having a loss-of-function mutation in one of the PEX genes listed in Table 2. According to another preferred aspect of the invention, the strains deficient in the beta-oxidation of fatty acids includes the strains having a loss-of-function mutation in one of the following genes: POX1, POX2, POX3, POX4, POX5, POX6, MFE1 [and] POT1. More preferably, the strains according to the invention include at least one loss-of-function mutation in at one of the genes POX1, POX2, POX3, POX4, POX5 and POX6. According to an even more preferred mode the strains according to the invention include mutations in each of the genes POX1, POX2, POX3, POX4, POX5 and POX6.

According to a particular embodiment, the invention therefore relates to a novel strain of yeast, particularly oleaginous yeast, specifically a mutant strain of Yarrowia lipolytica, which overexpresses the gene GPD1 and which comprises at least one loss-of-function mutation in one of the genes responsible for beta-oxidation of the fatty acids, said strain of yeast being capable of accumulating lipids. Said strain of yeast advantageously comprises at least one loss-of-function mutation in at least one of the genes chosen from among the genes PEX, POX, MFE1 and POT1. In a more particularly preferred manner, the POX genes are partially (POX2 to POX5) or totally (POX1 to POX6) inactivated in the mutant strain of the invention, said mutant strain being capable of accumulating lipids.

Besides the aforementioned loss-of-function mutations, which lead to a beta-oxidation deficiency, the strain of yeast according to the invention can comprise one or more additional mutations in at least one gene encoding an enzyme involved in the metabolism of fatty acids. This (these) additional mutation(s) can have the effect of further increasing the capacity of the strain to accumulate lipids. Alternatively, it (they) can alter the profile of the stored fatty acids. Thus, the present inventors have also shown that the expression of the genes TGL3 and TGL4 is increased when the POX genes are inactivated. The genes TGL3 and TGL4 encode lipases involved in the degradation of the fatty acids (Kurat et al., J. Biol. Chem., 281: 491-500, 2006). Without wishing to be limited by theory, it may be thought that the increased expression of these two genes undoubtedly serves to compensate for the absence of a functional beta-oxidation pathway for degrading fatty acids. Therefore, the invention also relates to a strain of yeast, preferably a strain of oleaginous yeast, specifically a mutant strain of Yarrowia lipolytica, which is deficient with regard to beta-oxidation and deficient with regard to the products of the genes TGL3 and TGL4; said strain overexpresses the gene GPD1 and is capable of accumulating lipids.

According to another preferred embodiment of the invention, the preferably oleaginous strain of yeast, more preferably a mutant Yarrowia lipolytica [strain] that is deficient with regards to beta-oxidation, further comprises a loss-of-function mutation in the GUT2 gene; said strain overexpresses the GPD1 gene and is capable of accumulating lipids.

It has also been shown that the inactivation of the gene YAL10B10153g, which encodes a Δ12 fatty acid desaturase, enables the proportion of C18:1 fatty acids (WO 2005/047485) to be increased. Therefore, the present invention also relates to providing a strain of yeast, in particular a mutant oleaginous yeast, specifically Yarrowia lipolytica, which is deficient with regard to beta-oxidation, which overexpresses the gene GSD1 and is capable of accumulating lipids, and which further comprises an inactivated YAL10B10153g gene.

According to another aspect, the preferably oleaginous mutant strain of yeast of the invention, specifically Y. lipolytica, which is deficient with regard to beta-oxidation, overexpresses the GPD1 gene and is capable of accumulating lipids, and further contains a gene the expression of which enables the fatty acid profile of said strain to be modified. It has indeed been reported that the ectopic expression of genes encoding certain desaturase enables polyunsaturated fatty acid profile to be modified in a strain of yeast, and in particular in Y. lipolytica. Thus, the expression of a Δ12 fatty acid desaturase enables obtainment of a larger quantity of fatty acids at C18:2 (WO 2005/047485). Likewise, the expression of a Δ8 desaturase or a M5 desaturase leads to a change in the fatty acid profile of Y. lipolytica (WO 2005/047480; WO 2006/012325). The invention therefore also relates to a mutant strain of yeast, in particular Y. lipolytica, which is deficient with regard to beta-oxidation, overexpresses the GPD1 gene and is capable of accumulating lipids, and which further expresses a gene encoding for an enzyme chosen from among a Δ8 desaturase, a Δ12 desaturase and a Δ15 desaturase. Said enzyme is preferably a Δ12 desaturase. More preferably yet, the gene encoding said Δ12 desaturase is the Y. lipolytica gene, the accession number of which is YALIOB10153g.

The invention also relates to a method for obtaining a mutant strain of yeast, said mutant strain being capable of accumulating lipids. In a preferred embodiment, the yeast is an oleaginous yeast. In a more preferable embodiment, the yeast is R. glutinis, R. tolurides or Yarrowia lipolytica. In a yet more preferable embodiment, the yeast is Yarrowia lipolytica.

The method according to the invention comprises at least two steps, said steps being capable of being carried out either simultaneously or consecutively. If the two steps are carried out one after the other, the order in which they are carried out does not matter. The method according to the invention therefore comprises the steps consisting in:
inactivating at least one gene controlling beta-oxidation, and
Transforming the strain of yeast by means of a polynucleotide enabling expression of the gene GPD1.
   The first step of the method according to the invention therefore consists in inactivating a gene responsible for beta-oxidation. As indicated above, these genes are also the genes POX, MFE1 and POT1 (Table 1) and the PEX genes (Table 2). The prior art also teaches various methods capable of enabling obtainment of strains of yeast, in particular an oleaginous yeast, specifically Yarrowia lipolytica, in which a gene is inactivated.

For example, reference may be made to the method called POP IN/POP OUT, which has been used with yeasts, in particular with Yarrowia lipolytica, in order to delete the genes LEU2, URA3 and XPR2, as described in the review by G. Barth and colleagues: (Yarrowia lipolytica, in: Nonconventional Yeasts in Biotechnology A Handbook (Wolf, K., Ed.), Vol. 1, 1996, pp. 313-388. Springer-Verlag). Said method consists in integrating a vector including a gene of interest deleted from the locus in question, and then in selecting the excision of said vector and identifying a clone which, by recombination, has eliminated the wild gene and preserved the mutated gene.

According to the invention, a method may preferably be used that results in the gene of interest being inactivated.

According to the invention, the term inactivation or invalidation of a gene of interest (the two terms as used in the present invention are synonymous and therefore have the same meaning), is understood to mean any method resulting in the non-expression of the native protein encoded by said gene of interest, via modification of the nucleotide sequence comprising said gene, so that, even though the translation thereof might be effective, it would not lead to the expression of the native protein encoded by the wild gene of interest.

According to the invention, a method is preferably used which results in the expression of the gene of interest being completely silenced. This can be carried out by completely deleting the gene of interest, by partially deleting the gene of interest, or by inserting one or more nucleotides into said gene of interest; said method used renders the gene of interest nonfunctional (an inactivated or invalidated gene of interest), at the very least encoding a protein that does not have the properties of said native protein.

In this way, a strain of yeast is obtained, which does not express the gene of interest and which, in the remainder of the present text, will be named “the gene-of-interest-defective strain”.

The SEP method can also be used (Maftahi et al., Yeast 12: 859-868, 1996) which was adapted to Yarrowia lipolytica for successively disrupting the POX genes (Wang et al., J. Bacteriol., 181: 5140-5148, 1999). This method is faster, but still requires the use of a marker enabling counter selection. According to the invention, the SEP/Cre method will advantageously be used, which was developed by Fickers et al. (J. Microbiol. Methods, 55:3: 727-737, 2003) and that is described in international application WO2006/064131. This is a fast method that does not require the use of a marker enabling counter selection.

This method consists in:

1) Selecting a gene of interest that one wishes to inactivate,
2) Constructing a disruption cassette by PCR (“Polymerase Chain Reaction”) or by cloning,
3) Introducing a selection marker containing recombination sequences on both sides (advantageously the loxP and/or loxR sequences or derivatives thereof) enabling same to be recombined in order to eliminate the marker (advantageously a loxP type sequence, which enables recombination via the Cre recombinase),
4) Selecting the strains with the gene of interest deleted (transformation and selection of the transformants) and verifying the deletion,
5) Transforming using a vector that enables expression of the recombinase (advantageously the Cre recombinase, which enables recombination of the loxP/loxR sequences and elimination of the marker),
6) Isolating a clone having the gene of interest deleted and having lost the recombinase expression plasmid.

The insertion cassette of step 2 includes a gene encoding a selection marker (selection gene), said gene preferably being flanked by the promoter and terminator regions of the gene of interest, so as to enable complete replacement of the gene of interest via homologous recombination. According to a particular embodiment, the selection gene is further flanked by one or more recombination sequences, said recombination sequences enabling a recombination thereof, which results in the elimination of the gene encoding the selection marker. Advantageously, the recombination sequence or sequences is (or are) one (or more) loxP sequences or one (or more) loxR sequences or (one or more) sequences derived from said recombination sequences, said derived sequence(s) having preserved the activity of the original recombination sequences. In this step, the gene encoding the selection marker can preferably be flanked by loxP type sequences which, via the Cre recombinase, recombine with one another while producing a plasmid that includes the sequence of the gene encoding said selection marker.

The introduction of the invalidation cassette of step 3 into the recipient strain of yeast can be carried out by any technique known to a person skilled in the art. As indicated above, reference can be made to G. Barth et al. (Yarrowia lipolytica, in: Nonconventional Yeasts in Biotechnology A Handbook (Wolf, K., Ed.), Vol. 1, 1996, pp. 313-388. Springer-Verlag).

The transformants expressing the selection marker are selected in step 4. The presence of the marker can be verified by any method known to a person skilled in the art, e.g., such as PCR or Southern blot hybridization.

In step 5, a plasmid enabling the expression of a recombinase is introduced into a transformant selected in the previous step. Preferably, the plasmid will be a carrier of the Cre recombinase gene (Sauer, Mol. Cell, Biol., 7: 2087-2096, 1987), which enables recombination of the loxP/loxR sequences and elimination of the marker. This technique is currently used by persons skilled in the art seeking to excise a specific integrated sequence (Hoess and Abremski, J. Mol. Biol., 181: 351-362, 1984).

Step 6 is a standard step for selecting a clone having excised the selection gene and therefore having selection marker absence phenotype.

The invention relates specifically to a method for obtaining a strain of yeast, particularly an oleaginous strain of yeast, specifically a mutant Yarrowia lipolytica strain, which does not express a gene controlling beta-oxidation, characterized in that:
in a first step, an invalidation cassette is constructed, which includes the promoter and terminator sequences of said yeast gene, in particular an oleaginous yeast, specifically Yarrowia lipolytica, flanking a gene encoding a selection marker (selection gene), said selection gene itself being flanked on both sides of the sequence thereof by one (or more) recombination sequence(s), said recombination sequences enabling recombination therebetween, which results in the elimination of said gene encoding the selection marker;
in a second step, said invalidation cassette obtained in step 1 is introduced into a strain of yeast, in particular an oleaginous strain of yeast and specifically Yarrowia lipolytica;
in a third step, a strain of yeast is selected from among the strains of yeast transformed in step 2, in particular an oleaginous strain of yeast [and] specifically Yarrowia lipolytica, which is defective with regard to the gene of interest, said strain having introduced the marker gene in place of said gene of interest via double cross-over, thereby resulting in an inactivated gene;
in a fourth step, the invalidation of said gene in said strain of yeast selected in step 3 is verified.

According to a variant of the invention, the method can also have 2 additional steps, namely:
a fifth step during which said strain selected in step 4 is transformed using a vector enabling the expression of a recombinase, so as to eliminate the gene expressing the selection marker;
a sixth step during which a strain of yeast is isolated, which is defective with regard to the gene and which no longer expresses the marker gene.

The method for inactivating a beta-oxidation gene can then be repeated so as to inactivate another gene, if necessary. A person skilled in the art will thus be able to inactivate as many genes as necessary, by simply repeating the SEP gene inactivation method. Said person can thus construct the mutant strains of yeast described above, which comprise several inactivated genes.

According to the invention, a strain of yeast that will not carry out the beta-oxidation of lipids may advantageously be used, e.g., a strain that will not express the genes responsible for the beta-oxidation of lipids, such as the POX, MFE1 or POT1 genes, advantageously a strain not expressing the POX gene, at the very least the POX2, POX3, POX4 and POX5 genes, preferably the POX1, POX2, POX3, POX4, POX5 and POX6 genes, e.g., such as the strains described in international application WO 2006/064131 published on Jun. 22, 2006, preferably the strains:
MTLY37 (Leu⁺, Ura⁺; Δpox5, Δpox2, Δpox3, Δpox4::URA3),
MTLY40 (Leu⁺, Ura⁻; Δpox5-PT, Δpox2-PT, Δpox3-PT, pox4::ura3-41),
MTLY64 (Leu⁻, Ura⁻; Δpox5, Δpox2, Δpox3, Δpox4,::ura3-41, leu2::Hyg),
MTLY66 (Leu⁻¹, Ura⁻; Δpox5, Δpox2, Δpox3, Δpox4::ura3-41, Δleu2),
MTLY82 (Leu⁻, Ura⁻; Hyg⁻; Δpox5, Δpox2, Δpox3, Δpox4::ura3-41, Δleu2, Δpox1),
MTLY86 (Leu⁻, Ura⁻; Δpox5; Δpox2, Δpox3, Δpox4::ura3-41, Δpox1),
MTLY92 (Leu⁻, Ura⁻; Hyg⁺; Δpox5, Δpox2, Δpox3, Δpox4::ura3-41, Δleu2, Δpox1, pox6::Hyg),
MTLY95a (Leu⁻, Ura⁻; Δpox5, Δpox2, Δpox3, Δpox4::ura3-41, Δleu2, Δpox1, Δpox6)

In another aspect of the invention, a strain of yeast may be used such as those described in PCT application WO 2010/004141 published on Jan. 14, 2010. For example, the following strains may be used:
JMY1351 (Leu⁻, Ura⁺, Δpox5, Δpox2, Δpox3, Δpox4::ura3-41, Δleu2, Δpox1, Δpox6, Δgut2)
JMY1393 (Leu⁻, Ura⁻, Δpox5, Δpox2, Δpox3, Δpox4::ura3-41, Δpox1, Δpox6, Δgut2).

In yet another aspect of the invention, the strains may be used that are described in the publications Béopoulos et al. (Appl. Environ. Microbiol., 74(24): 7779-7789. 2008) and Wang et al (J. Bacteriol., 181: 5140-5148, 1999).

In a second step of the method according to the invention, the GPD1 gene is overexpressed in one of the beta-oxidation-deficient genes obtained in the preceding step.

The GPD1 gene encodes for the glycerol-3-phosphate dehydrogenase catalyzing the synthesis reaction of glycerol-3-phosphate from DHAP. The GPD1 gene can be overexpressed by any manner known to a person skilled in the art.

To accomplish this, each copy of the GPD1 open reading frame is placed under the control of appropriate regulatory sequences. Said regulatory sequences include promoter sequences placed upstream (at 5′) from the GPD1 open reading frame, and terminator sequences placed downstream (at 3′) from the GPD1 open reading frame.

The promoter and terminator sequences used preferably belong to different genes, so as to minimize the risks of undesirable recombination in the genome of the Yarrowia strain.

Such promoter sequences are well known to a person skilled in the art and can, in particular, correspond to inducible and constitutive promoters. As examples of promoters that can be used in the method of the invention, reference can be made in particular to the promoter of a Yarrowia lipolytica gene that is strongly suppressed by glucose and that can be induced by fatty acids or triglycerides, such as the POX2 promoter of the acyl-CoA oxidase gene of Yarrowia lipolytica and the promoter of the LIP2 gene described in PCT application WO 01/83773. It is also possible to use the FBA/gene promoter of the fructose-bisphosphate aldolase gene (US 2005/0019297), the GPM promoter of the phosphogylcerate mutase gene (WO 2006/0019297), the YAT1 gene promoter of the ammonium transporter gene (US 2006/0094102 A1), the GPAT gene promoter of the glycerol-3-phosphate O-acyltransferase gene (US 2006/0057690 A1), the TEF gene promoter (Muller et al., Yeast, 14: 1267-1283, 1998; US 2001/6265185), the hp4d hybrid promoter (WO 96/41889) or even the XPR2 hybrid promoters described in Mazdak et al. (J. Mol. Microbiol. Biotechnol., 2(2): 207-16, 2000).

Such terminator sequences are likewise well-known to a person skilled in the art, and, as examples of terminator sequences that can be used in the method according to the invention, reference can be made to terminator sequence of the PGK1 gene, and the terminator sequence of the LIP2 gene described in PCT application WO 01/83773.

The overexpression of GPD1 can be obtained by replacing the sequences controlling the expression of GPD1 by regulatory sequences enabling stronger expression, such as those described above. A person skilled in the art can thus replace the copy of the GPD1 gene in the genome, as well as the specific regulatory sequences thereof, by transforming the mutant strain of yeast with a linear polynucleotide including the GPD1 open reading frame under the control of regulatory sequences such as those described above. Said polynucleotide is advantageously flanked by sequences that are homologues of sequences situated on each side of the chromosomal GPD1 gene. Insofar as this recombination event is rare, selection markers are inserted between the sequences ensuring recombination so that, after transformation, it is possible to isolate the cells where integration of the fragment occurred, by highlighting the corresponding markers.

The overexpression of GPD1 is obtained by introducing into the strain of yeast according to the invention supernumerary copies of the GPD1 gene under the control of regulatory sequences such as those described above. Said additional copies of GPD1 can be carried by an episomal vector, i.e., one capable of replicating in the yeast.

Said copies are preferably carried by an integrative vector, i.e., being integrated at a specific location in the genome of the yeast (Mazdak et al., J. Biotechnol., 109(1-2): 63-81, 2004). In this case, the polynucleotide comprising the GPD1 gene under the control of regulatory regions is integrated by targeted integration.

Targeted integration of a gene in the genome of a yeast is a technique frequently used in molecular biology. In this technique, a DNA fragment is cloned in an integrative vector introduced into a cell being transformed, which DNA fragment is then integrated by homologous recombination in a targeted region of the recipient genome (Orr-Weaver et al., Proc. Natl. Acad. Sci. USA, 78: 6354-6358, 1981). Such transformation methods are well known to a person skilled in the art and are described, in particular, in Ito et al. (J. Bacteria 153: 163-168, 1983), in Klebe et al. (Gene, 25: 333-341, 1983) and in Gysler et al. (Biotechn. Techn., 4: 285-290, 1990). Insofar as this recombination event is rare, selection markers are inserted between the sequences ensuring recombination so that, after transformation, it is possible to isolate the cells where integration of the fragment occurred, by highlighting the corresponding markers.

They can also be carried by PCR fragments the ends of which have homology with a specific locus of the yeast, thus enabling said copies to be integrated into the genome of the yeast by homologous recombination.

Any transfer method known to a person skilled in the art can be used to introduce the invalidation cassette 1 into the strain of yeast. Preferably, use can be made of the lithium acetate and polyethylene glycol method (Gaillardin et al., Curr. Genet., 11: 369-375, 1987; Le Dall et al., Curr. Genet., 26(1): 38-44, 1994).

According to the invention, it is possible to use any selection method known in the prior art, which is compatible with the gene (or genes) used, any strain expressing the selected marker gene potentially being a strain of yeast defective with regard to the GUT2, URA3 or LEU2 gene.

Selection markers enabling auxotrophy complementation, likewise commonly called auxotrophy markers, are well-known to a person skilled in the art.

The URA3 selection marker is well-known to a person skilled in the art. More specifically, a strain of Yarrowia lipolytica, the URA3 gene of which (said sequence is also accessible by the accession number YALIOE26719g at the address: http://cbilabri.fr/Genolevures/index.php#), encodes for the orotidine-5′-phosphate decarboxylase, is inactivated (e.g., by deletion), will not be capable of growing in a medium not supplemented with uracil. Integration of the URA3 selection marker into this strain of Yarrowia lipolytica will then enable the growth of this strain to be restored in a uracil-free medium.

The LEU2 selection marker, described in particular in U.S. Pat. No. 4,937,189, is likewise well-known to a person skilled in the art. More specifically, a strain of Yarrowia lipolytica, of which the LEU2 gene (YALIOE26719g) encodes for the β-isopropylmalate dehydrogenase, is inactivated (e.g., by deletion), and will not be capable of growing in a medium not supplemented with leucine. As previously, integration of the LEU2 selection marker will then enable the growth of this strain to be restored in a medium not supplemented with leucine.

The ADE2 selection marker is likewise well-known to a person skilled in the art, in the field of yeast transformation. A strain of Yarrowia, of which the ADE2 gene (YALIOB23188g) encodes for the phosphoriboxylaminoimidazole carboxylase, is inactivated, and will not be capable of growing in a medium not supplemented with adenine. Here again, integration of the ADE2 selection marker in this strain of Yarrowia lipolytica will then allow one to restore the growth of this strain on a medium not supplemented with adenine.

The invention also relates to the use of a mutant strain of yeast, especially an oleaginous yeast, in particular Yarrowia lipolytica, for synthesizing lipids, especially free fatty acids and triacylglycerols. In a more especially preferred aspect, the invention relates to a strain of yeast, specifically a mutant oleaginous yeast, in particular Yarrowia lipolytica, which is deficient with regard to beta-oxidation and which overexpresses GPD1, as described above, for synthesizing free fatty acids and triacylglycerols.

The present invention also relates to a lipid-synthesizing method in which:
in a first step, a strain of yeast according to the invention is cultivated in an appropriate medium, and
in a second step, the lipids produced by the culture of step 1 are harvested.

In addition to the preceding arrangements, the present invention likewise includes other characteristics and advantages, which will emerge from the following examples and figures, and which must be considered as illustrating the invention without limiting the scope thereof.

### FIGURES LEGEND

FIG. 1. Diagram of the various fatty acid synthesis pathways, storage and degradation of the neutral lipids and the connection thereof with the glyoxylate and TCA cycles. The dotted arrows indicate hypothetical enzyme reactions. The dashed arrows represent simplified reactions. The proteins encoded by the genes between parentheses have been shown as being associated with the lipid bodies (LB) in Yarrowia lipolytica, DAG, diacylglycerol; DHA, dihydroxyacetone; DHAP, dihydroxyacetone phosphate; FFA, free fatty acid; GAD3P, glyceraldehydes-3-phosphate; Gly-3P, glycerol-3-phosphate; LPA, lysophosphatidic acid; MAG, monoacylgylcerol, PA, phosphatidic acid; PL, phospholipids; SE, steryl ester; TAG triacylglycerol, TCA cycle, tricarboxylic acid cycle.

FIG. 2. Analysis of the expression of the genes involved in homeostasis of the triacylglycerols in various strains of Yarrowia lipolytica.

The expression of the various genes was measured by qRT-PCR using specific primers for each of the genes and was related to the quantity of transcripts of the ACT1 gene encoding for actin (YALIOD08272g). These results correspond to the mean standard deviation±derived from three separate experiments.

FIG. 3. Accumulation of the lipids in different strains of Yarrowia lipolytica. The yeasts were cultivated for 24 hours in YNBD2 (A) or in YNBDO.503 (B). The black bars represent the strains overexpressing GPD1; the white bars represent the parent strains. Extraction of the fatty acids was reproduced using three separate specimens.

FIG. 4. Growth of various strains in a YNBDO.503 medium. The growth of the WT and mutant strains listed in Table 1 were followed over time. These results correspond to the mean standard deviation±derived from three separate experiments.

FIG. 5. Phenotypes of the LBs in various strains of Yarrowia lipolytica. The morphologies of the LBs of the WT, Δgut2, Δpox1-6, Δgut2Δpox1-6, WT GPD1⁺, Δgut2 GPD1⁺, Δpox1-6 GPD1⁺ and Δgut2Δpox1-6 GPD1⁺ strains are shown. The optical microscopy images are presented in the left-hand columns and the corresponding fluorescence microscopy images are presented on the right-hand side. Fluorescence was obtained after coloring with the neutral green stain LipidTox™. The strains were cultivated for 24 hours in a YNDBO.503 medium.

FIG. 6. Triacylglycerol and free fatty acid content as fractions of the total lipids.

This graph shows the total accumulation of lipids after 24 hours of growth in a YNDBO.503 medium, as a percentage of the dry weight of the cells. The lipids were fractionated into triacylglycerols and free fatty acids by solid phase extraction, and quantification was carried out by gas chromatography. Extraction of the fatty acids was reproduced using two separate specimens; a representative result of the two experiments is shown.

FIG. 7A. Sequence of the GPD1⁺ gene; B. Sequence of the Gpd1 polypeptide.

### EXAMPLES

### Material and Methods

### Yeast Strains, Growth and Culture Conditions

The strains of Yarrowia lipolytica used in this study are derived from the wild strain (wild-type; WT) of Yarrowia lipolytica W29 (ATCC20460) (Table 3). The auxotrophic strain Po1d (Leu “Ura”) was described by Barth and Gaillardin (1996. Yarrowia lipolytica, p. 313-388, in K. Wolf, K. D. Breunig, and G. Barth (ed.), Nonconventional yeasts in biotechnology, vol. 1. Springer-Verlag, Berlin, Germany). The auxotrophic strain MTY94, in which the six POX genes encoding acyl-CoA oxidases (Aox) were inactivated, has been described previously (Beopoulos et al., Appl. Environ. Microbiol., 74(24): 7779-7789, 2008). The auxotrophic strain JMY1367, in which the six POX genes encode the acyl-CoA oxidases (Aox) and the GUT2 gene, which encodes the glycerol-3-phosphate dehydrogenase, were inactivated, is derived from the strain JMY1367 (Beopoulos et al., Appl. Environ. Microbiol., 74(24): 7779-7789, 2008). To obtain JMY1346 and JMY1367, the URA3 marker was excised from JMY1202 and JMY1351, respectively, as previously described (Beopoulos et al., Appl. Environ. Microbiol., 74(24): 7779-7789, 2008). The strains used in this study are listed in Table 3.

The medium and culture conditions for Escherichia coli are described in Sambrook et al. (1989. Molecular cloning: a laboratory manual, 2^{nd} ed., Cold Spring Harbor Laboratory Press, Cold Spring Harbor, N.Y.), and those for Yarrowia lipolytica are described in Barth and Gaillardin (1996. Yarrow lipolytica, p. 313-388., in K. Wolf, K. D. Breunig and G. Barth (ed.), Nonconventional yeasts in biotechnology, vol. 1. Springer-Verlag, Berlin, Germany). The rich medium (YPD) and the minimal medium with glucose (YNB) were prepared as previously described (Mlickova et al., Appl. Environ. Microbiol., 70: 3918-3924, 2004).

The minimum medium YNB contains 0.17% YNBww (YNB medium without amino acids and ammonium sulfate; Difco, Paris, France), 0.5% NH₄Cl, 0.1% yeast extract (Bacto-DB) and 50 mM of a phosphate buffer at pH 6.8. This medium can be supplemented with 0.2% casamino acids (Difco, Paris, France) and/or 0.1 g/L uracil.

The medium supplemented with a carbon source are:
the YNBD medium (2% glucose, Merck, Fontenay-sous-Bois Cedex, France);
the YNBG medium (2% glycerol, Merck);
the YNBDO medium (medium supplemented with 3% of 60% pure Merck oleic acid);
the YNBOu.p. medium (medium supplemented with 3% of 98% pure Flucka oleic acid);
the YNBO medium (YNBDO.503) used for monitoring optimum accumulation and remobilization of the fatty acids consists of the YNB medium supplemented with 0.5% glucose and 3% oleic acid;
the YP₂D₄O₃ medium, used to optimize the accumulation of lipids, contains yeast extract (1%), peptone proteose (2%), glucose (4%) and oleic acid (3%). Uracil (0.1 g/L) and leucine (0.2 g/L) were added as needed. For the solid media, 1.5% agar was added. The oleic acid is emulsified by ultrasonication in the presence of 0.02% Tween 40 (Mlickova et al., Appl. Environ. Microbiol., 70: 3918-3924, 2004).

As a general rule, the cultures were made as follows: using a YPD dish, a first preculture is inoculated into YPD medium (15 ml in 50-ml Erlenmeyer flasks at 170 rpm, at 29° C. for 6 hours). The cells were used to inoculate a preculture with YNBD medium (50 ml in a 500-ml Erlenmeyer flask at 170 rpm, at 28° C. for one night).

For the culture, exponential growth cells were harvested the following day by centrifugation of the preculture, [then] washed and resuspended in a fresh YNB medium at an optical density at 600 nm of 0.5.

In order to determine the cell growth, the cultures were centrifuged at 3,000 g for 5 min., and the cell pellet was washed twice with equal volumes of SB solution (9 g/L of NaCl—0.5% BSA). The resulting biomass was determined by measuring the optical density at 600 nm and by estimating the dry weight of the cells after lyophilization.

### General Genetic Techniques

The general techniques of molecular genetics were used, as described in Sambrook et al. (1989, Molecular cloning: a laboratory manual, 2^{nd} ed. Cold Spring Harbor Laboratory Press, Cold Spring Harbor, N.Y.). The restriction enzymes used come from Eurogentec SA (Liege, Belgium).

The yeast cells were transformed according to the lithium acetate method described in Le Dall et al. (Curr. Genet., 26(1): 38-44, 1994). The genomic yeast DNA (wild or transformed) was prepared as described by Querol et al., (Appl. Environ. Microbiol., 58(9): 2948-2953, 1992). The PCR amplifications were carried out on an Eppendorf 2720 thermocycler, with either Taq polymerase DNA (Promega, Madison, Wis., USA) or with Pfu polymerase DNA (Stratagene, La Jolla, Calif.). The GPD1 gene was amplified using the primers described in Table 4. The PCR fragments were purified using the QIAGEN purification kit (Qiagen, Hilden, Germany), and the DNA fragments were recovered from agarose gels, using a QIAquick Gell Extraction kit Qiagen, Hilden, Germany). The Staden program package was used for analyzing the sequences (Dear. And Staden, Nucleic Acid Res., 19: 3907-3911, 1991).

### Preparation of the RNAs and Quantification of the Transcripts

The biological specimens were frozen in liquid nitrogen and kept at −80° C. The RNAs were extracted with the RNeasy Mini Kit (Qiagen, Courtaboeuf, France) while following the manufacturer's instructions. For the Q-PCR experiments, the total RNA for each specimen was treated with Dnase I (Ambion). The absence of genomic DNA in the RNA preparations was verified by PCR, by using the total RNAs treated with Dnase I as a matrix and the Taq (Promega, Madison, Wis.) as an enzyme. The quality of total RNAs was controlled using the Bioanalyzer Agilent 2100, the reactants from the Agilent RNA 6000 Nano kit and RNA chips. The total RNAs treated with Dnase I (1 μg) were treated with the reverse transcriptase Thermoscript RT (Invitrogen, Carlsbad, USA), while following the manufacturer's instructions. The Q-PCR experiments were carried out by means of the LightCycler 1.5 (Roche, Meylan, France), using the LightCycler Fast Start DNA Master Sybr Green kit (Roche, Meylan, France), while following the manufacturer's instructions. The expression of each gene was quantified using the 2ACT method, by calibrating with ACT1, which encodes actin. The amplification reaction was as follows: 95° C. for 8 min., 95° C. for 10 sec., 60° C. for 6 sec., and 72° C. for 10 sec. (45 cycles), 95° C. for 15 sec., 60° C. for 15 sec. and 95° C. for 15 sec., 40° C. for 5 min. The Measurement of the expression of each gene via PCR was reproduced on three separate specimens. The primers used for the Q-PCR are indicated in Table 4.

### Fluorescence Microscopy

In order to view the lipid bodies (LB), the fluorochrome LipidTOX™ Green (2.5 mg/ml ethanol; Invitrogen) was added to a cell suspension (A₆₀₀=5) and incubated for 10 min. at ambient temperature. Microscopy was carried out with an AXIO Imager M1 fluorescence microscope (Zeiss, Le Pecq, France) with a 100× immersion objective. The AxioVision Rel. 4.6 program was used for image acquisition.

### Determination of the Lipids

The lipids of a quantity of cells greater than 5 mg were extracted either by means of the Folch et al. procedure (J. Biol. Chem. 226(1): 497-509, 1957) for TLC analysis, or directly converted into the methyl esters thereof by using lyophilized cells, according to Browne et al., (Anal. Biochem., 152(1): 141-145, 1986) for gas chromatography analysis. Complete transmethylation was verified by means of a BF₃ in methanol method (Athenstaedt et al., J. Bacteriol., 181(20): 6441-6448, 1999) and on TLC plates.

Gas chromatography analysis of the fatty acid methyl esters was carried out using a Varian 3900 provided with a flame ionization detector and a Varian FactorFour vf-23 ms column (3 pA at 260° C. [30 min., 0.25, 0.25 μm]).

The free fatty acids were identified by comparison with standard fatty acid methyl esters (Fatty Acid Methyl Ester, FAME, Cayman, Supelco, Sigma, France) and quantified by the internal standard method, by adding 50μ of commercial (Sigma) C17:0.

### Analysis of the Lipid Bodies

The lipid bodies were analyzed by microscopic observation of a growing cell culture, as described above. Differential phase contrast objectives (Nomarski) were used to obtain the transmitted images.

### Analysis of Classes of Lipids

The total lipids were fractionated into triacylglycerols and free fatty acids, in order to quantify the lipids, using an Isolute SPE Aminoprpyl column (IST France, Paris, France), by following the teaching of Laffargue et al. (Plant Physiol. Biochem., 45(3-4): 250-257, 2007). The column was conditioned 3 times with 3 ml of normal hexane at a normal flow rate. One ml of all of the lipids extracted using the Folch et al. method (J. Biol. Chem., 226(1): 497-509, 1957) in CHCl₃ was loaded into the column and the fraction of neutral lipids was collected.

Complete elution of the neutral lipids was carried out by washing the column 3 times with 3 ml of CHCl₃/isopropanol (2/1). The fraction of free fatty acids was collected by washing the column 3 times with 3 ml of ET₂O/2% acetic acid at a normal flow rate. The fraction solvent was evaporated under a direct nitrogen flow and transmethylation was followed for by gas chromatography analysis (Laffargue et al., Plant Physical. Biochem., 45(3-4): 250-257, 2007).

TLC plates were used for the verification extractions. The effectiveness of the procedure was likewise verified by comparison with the gas chromatography profiles of the fractionated or non-fractionated specimens.

### TLC Separation of the Lipids

TLC plates (silica G60, 20×20 cm, 0.25 mm thick) (Merck, Germany) were used. The various classes of lipids were separated using a double development solvent system: System A (migration half-plate): petroleum ether/methyl ether/acetic acid; 20/20/0.8 (v/v/v); System B (the entire migration plate): petroleum ether/Et₂O 49/1 (v/v). A 5% solution of phosphomolybdic acid was sprayed over the plates and the bands of lipids were revealed after 10 min. at 105° C.

### NMR Spectroscopy

PCA (perchloric acid) extracts were prepared from 1 g dry weight of Yarrowia lipolytica by following the method described in Auber et al. (J. Cell Biol., 133: 1251-1263, 1996). The values are expressed in mg g⁻ of dry weight of yeast. The spectra were recorded on a Bruker RMN spectrometer (AMX 400, wide bore; Bruker, Billerica, Mass.) provided with a 10 mm or 25 mm multinuclear probe set at 161.9 or 100.6 MHz for the ³¹P— and ¹³C analyses. Deuterium (²H₂O) resonance was used as a reference signal. For the measurements, the acquisitions of ¹³C-NMR and ³¹P-NMR were carried out as described previously (Dulermo et al., New Phytol. 183: 1149-1162, 2009). The solvent used for the NMR come from Sigma-Aldrich and Leman (Archamps, France).

### Results

### Construction of Strains

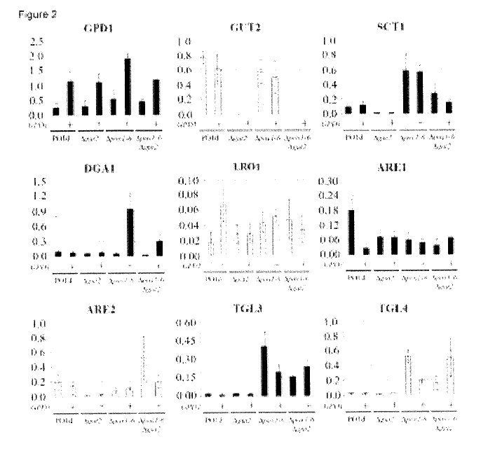

The GPD1 gene of Yarrowia lipolytica was overexpressed in WT, Δgut2, Δpox1-6, and Δgut2Δpox1-6 of Yarrowia lipolytica. These strains were transformed with an expression cassette containing GPD1 under the control of the constitutive promoter TEF (Muller et al., Yeast, 1.4: 1267-1283, 1998). The transformants containing the expression cassette were identified via PCR (data not shown). Using quantitative RT-PCR, it was observed that the GPD1 gene was expressed at the same levels in the various transformants (FIG. 2). In the GPD1⁺ strains, the expression of GPD1 is increased by a factor of 5 to 6 relative to the wild type. Identical results were obtained by analyzing two transformants from each GPD1⁺ strain.

### The Overexpression of GPD1⁺ Results in an Increase in the Quantity of Cellular Lipids

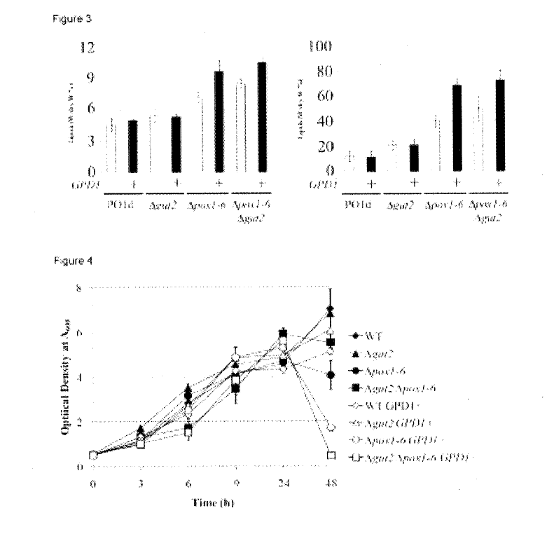

First of all, the ability of various strains to synthesize lipids de novo was studied after 24 hours of cultivation in YNBD2: at 24 hours, the overexpression of GPD1 had no effect in the wild-type and in Δgut2, whereas the quantity of cellular lipids increased by 34% and 35% in the Δpox1-6 GPD1⁺ and Δgut2Δpox1-6 GPD⁺ strains, respectively, by comparison with the isogenic parental strains (FIG. 3A).

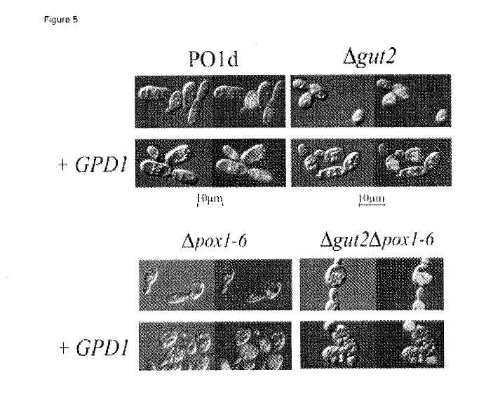

In a second phase, the accumulation capacity of the lipids of the various strains was analyzed. To do so, the strains were cultivated in YNBDO.503. While all of the transformants had the same growth rate as the wild-type in YNBD2 (data not shown), this was not the case in YNBDO.503. In this particular medium, the growth of Δgut2Δpox1-6 GPD1⁺ and, to a lesser extent, Δpox1-6 GPD1⁺, is dramatically affected between 24 and 48 hours of cultivation, whereas this is not the case for the other strains. In particular, the absorption of these two cultures drops sharply between 24 and 48 hours, thereby indicating cell lysis (FIG. 4). The death of these cells is probably due to an excessive accumulation of lipids. For this reason, the accumulation of lipids was analyzed in various transformants after 24 hours of cultivation in YNBDO.503 (FIG. 3B). Just as for the de novo synthesis of lipids in YNBD2, the accumulation of lipids is only increased in the Δpox1-6 GPD1⁺ and Δgut2Δpox1-6 GPD⁺ strains, by 70% and 47% relative to the isogenic parental strains thereof, respectively. The accumulation of lipids accounts for more than 65-75% of the dry weight of these strains, i.e., 5 times the quantity of lipids in the wild strain at 24 hours. The overexpression of GPD1 has no effect on the fatty acid profile of the transformants (Table 5).

### Microscopic Analysis of Strains Overexpressing GPD1

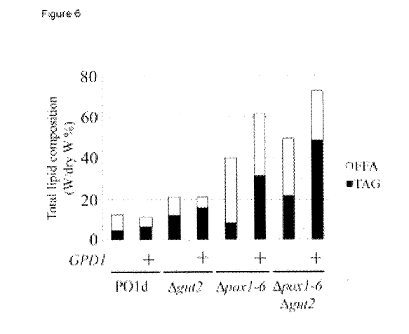

The overexpression of GPD1 results in an increase in the lipid content in the Δpox1-6 and Δgut2Δpox1-6 strains. In S. cerevisiae, the majority of the stored lipids are neutral lipids, i.e., triacylglycerols and sterol esters. In Yarrowia lipolytica, the neutral lipids are stored in a specialized compartment, the lipid body (LB) (Czabany et al., Biochim. Biophys. Acta, 1771: 299-309, 2007). In order to estimate the effect of the overexpression of GPD1 on the lipid bodies of Yarrowia lipolytica, the cells of various strains were analyzed under a microscope, after 24 hours of growth on YNBDO.503. The Δgut2, Δgut2 GPD1′, Δpox1-6, Δpox1-6 GPD1⁺, Δgut2Δpox1-6 and Δgut2Δpox1-6 GPD1⁺ cells showed very significant lipid bodies (FIG. 5). No difference could be detected between WT and WT GPD1⁺, as well as between Δgut2 and Δgut2 GPD1′. On the other hand, the Δpox1-6 GPD1⁺ and Δgut2Δpox1-6 GPD1⁺ strains had the largest and most numerous lipid bodies. The overexpression of GPD1 not only results in an increase in the quantity of cellular lipids but also appears to affect the number and size of the lipid bodies in Yarrowia lipolytica.

### The Overexpression of GPD1 Results in an Increase in the Fraction of Triacylglycerols

In order to quantify the fraction of triacylglycerols in the GPD1⁺ strains, the neutral lipids and free fatty acids were fractionated with an SPE Aminopropyl column. The fractions of triacylglycerols and free fatty acids were then analyzed by gas chromatography.

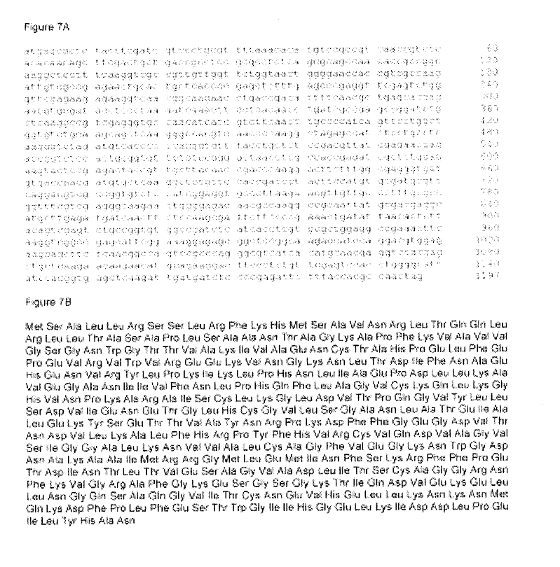

The quantity of triacylglycerols was analyzed in all of the strains after 24 hours of cultivation in YNBDO.503 (FIG. 6).

The deletion of GUT2 and/or the overexpression of GPD1 lead to an increase in the fraction of triacylglycerols. The level of triacylglycerols is increased by a factor of 1.3, 2.4 and 3.2 in the WT GPD1⁺, Δgut2 and Δgut2 GPD1⁺ strains, respectively, relative to the wild strain. The inactivation of the POX1-6 genes has little effect on the level of triacylglycerols, with an increase by a factor of 1.7 in comparison with the wild strain. On the other hand, the association of the POX1-6 mutations with the inactivation of GUT2 and/or the overexpression of GPD1 has a major effect on the quantity of triacylglycerols, with triacylglycerol levels in the Δgut2Δpox1-6, Δpox1-6 GPD1⁺ and Δgut2Δpox1-6 GPD1⁺ strains reaching 4.6 and 10 times the level observed in the wild strain, respectively. In the Δgut2Δpox1-6 GPD1⁺ strain, the fraction of triacylglycerols accounts for more than 50% of the dry weight.

### NMR Metabolic Analyses of the Various Strains

The results obtained clearly show that the overexpression of GPD1 dramatically affects the synthesis of the triacylglycerols. This could be explained by an increase in the concentration of G3P. In order to verify whether the inactivation of GUT2 or the overexpression of GPD1 affects the synthesis of G3P, the parent strains and GPD1⁺ were cultivated on YNBDO.503, and the metabolites were analyzed by NMR. The analysis of the spectra of ³¹P indicates that the concentration of G3P is increased by 1.5 to 5.6 times, in the strains where GUT2 is inactivated or in the strains overexpressing GPD1, relative to the wild strain (Table 6). When GUT2 is functional, the overexpression of GPD1 has only a small effect on the level of G3P. For example, the concentration of G3P is increased by only 50% in the WT GPD1⁺ and Δpox1-6 GPD1⁺ strains, in comparison with the parental strains. The level of G3P is slightly higher in the Δpox1-6 strain compared with the wild strain. The concentration of G3P is increased by a factor of three in the Δgut2 and Δgut2Δpox1-6 strains, relative to the wild strain. The maximum concentration of G3P is obtained in the Δgut2 GPD1⁺ strains. Additionally, the G3P concentration in the Δpox1-6 GPD1⁺, and surprisingly in the Δgut2Δpox1-6 GPD1⁺ strain, is only twice the concentration of the wild type. It therefore appears that the deletion of GUT2 or the overexpression of GPD1 have different consequences with regard to the synthesis of G3P.

G3P is an intermediary metabolite in the synthesis of glycerol. While the concentration of G3P is increased in the Δgut2 and GPD1⁺ strains, the concentration of glycerol is reduced in these strains and even more dramatically in the Δgut2 GPD1⁺, Δpox1-6 GPD1⁺, Δgut2Δpox1-6 and Δgut2Δpox1-6 GPD1⁺ strains.

In Δgut2 GPD1⁺, the strain accumulating the highest levels of G3P, a doubling and tripling of the respective concentrations of glycerol phosphorylcholine (GPC) and glycerol phosphoryletanolamine (GPE) was observed. Since the NMR was not sufficiently sensitive, it was not possible to detect more than three intermediates for the glyoxylate/TCA cycles (malate, citrate and succinate); a sharp reduction was observed for each of the three compounds in the POX strains or genes, or the GUT2 gene was inactivated. Additionally, the overexpression of GPD1⁺ in these strains accentuates the disappearance of these compounds.

### Modification of the Expression of the Genes Involved in the Metabolism of Triglycerides

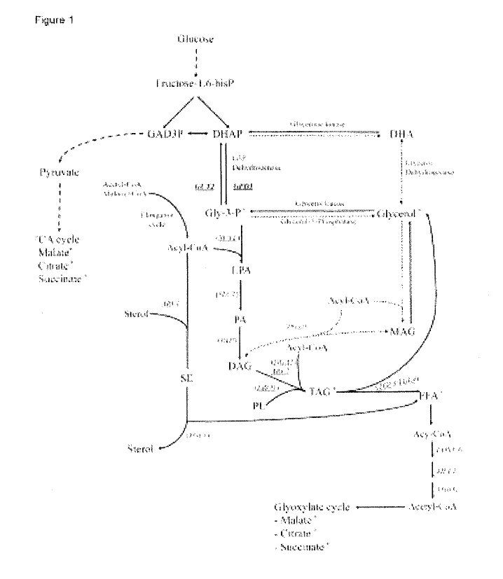

By means of quantitative RT-PCR, we have analyzed the expression of GUT2 and the genes involved in the synthesis of triacylglycerides, such as SCT1, DGA1, LRO1, ARE1 and ARE2, which encode acyltransferases (Beopoulos et al., Prog. Lipid Res., 48: 375-387, 2009; FIG. 1). The overexpression of GPD1 has no effect on the expression of GUT2. In the same way, the deletion of GUT2 has no effect on the expression of GPD1. On the other hand, a change in the expression of the genes involved in the metabolism of G3P has a significant effect on DGA1. In the Δpox1-6 GPD1⁺ and Δgut2Δpox1-6 GPD1 strains, the expression of DGA1 is increased by 15 and 10 times, respectively, relative to the parental strains. Surprisingly, the deletion of the POX genes results in a large increase in the expression of SCT1, whereas the deletion of GUT2 has an antagonistic effect and the overexpression of GPD1 has no effect. The expression of ARE1 is reduced in the Δgut2 strain only. The expression of LRO1, which is weakly expressed, is not modified in the various strains.

The accumulation of triacylglycerols depends solely on the synthesis activities. For this reason, the degradation processes must have a significant role in the homeostasis of the triacylglycerols. The first step in the catabolism of the triacylglycerols is catalyzed by intracellular triacylglycerol lipases (Czabany et al., Biochim. Biophys. Acta., 1771: 299-309, 2007; FIG. 1). The Yarrowia lipolytica yeast has intracellular triacylglycerol lipases that are homologues of the Tg13 and Tg14 proteins of S. cerevisiae, but no homologue of Tg15 (Beopoulos et al., Appl. Environ. Microbiol., 74: 7779-7789, 2008). The expression of the TGL (TGL3 and TGL4) genes was analyzed by quantitative RT-PCR. These two genes have similar expression profiles. TGL3 and TGL4 are strongly expressed in the strains in which the POX genes are inactivated. However, the deletion of GUT2 or the overexpression of GPD1 has a negative effect on the expression of same. For example, the expression of TGL3 is increased 20 times in the Δpox1-6 strain and only 7 times in the Δpox1-6 Δgut2 strain, relative to the wild strain. Overall, these results clearly show that modifying the metabolism of the G3P or POX genes affects the expression of the genes involved in the homeostasis of triacylglycerols.

TABLE 1
Genes involved in the metabolism of fatty acids in yeasts, particularly in
Yarrowia lipolytica. The sequences are accessible by their names or accession
numbers at the address: http://cbi.labri.fr/Genolevures/index.php#.

| Gène | Nom | N° EC | Fonction |
|---|---|---|---|
| GUT1 | YALI0F00484g | EC 2.7.1.30 | Glycerol kinase |
| GPD1 | YALI0B02948g | EC 1.1.1.18 | Glycerol-3-phosphate dehydrogenase |
| | | | (NAD(+)) |
| GUT2 | YALI0B13970g | EC 1.1.99.5 | Glycerol-3-phosphate dehydrogenase |
| SCT1 | YALI0C00209g | EC 2.3.1.15 | Glycerol-3-phosphate acyltransferase |
| SLC1 | YALI0E18964g | EC 2.3.1.51 | 1-acyl-sn-glycerol-3-phosphate |
| | | | acyltransferase |
| DGA1 | YALI0E32769g | EC 2.3.1.20 | Diacylglycerol acyltransferase |
| LRO1 | YALI0E16797g | EC 2.3.1.158 | Phospholipid: diacylglycerol |
| | | | acyltransferase |
| TGL3 | YALI0D17534g | EC 3.1.1.3 | Triacylglycerol lipase |
| TGL4 | YALI0F10010g | EC 3.1.1.3 | Triacylglycerol lipase |
| ARE1 | YALI0F06578g | EC 2.3.1.26 | Acyl-CoA: sterol acyltransferase |
| ARE2 | YALI0D07986g | EC 2.3.1.20 | Diacylglycerol acyltransferase |
| TGL1 | YALI0E32035g | EC 3.1.1.13 | Cholesterol esterase |
| POX1 | YALI0E32835g | EC 6.2.1.3 | Acyl-coenzyme A oxidase |
| POX2 | YALI0F10857g | EC 6.2.1.3 | Acyl-coenzyme A oxidase |
| POX3 | YALI0D24750g | EC 6.2.1.3 | Acyl-coenzyme A oxidase |
| POX4 | YALI0E27654g | EC 6.2.1.3 | Acyl-coenzyme A oxidase |
| POX5 | YALI0C23859g | EC 6.2.1.3 | Acyl-coenzyme A oxidase |
| POX6 | YALI0E06567g | EC 6.2,1.3 | Acyl-coenzyme A oxidase |
| MFE1 | YALI0E15378g | EC 4.2.1.74 | Multi-functional beta oxidation protein |
| POT1 | YALI018568g | EC 2.3.1.16 | Peroxisomal Oxoacyl Thiolase |

Key to Table 1:
Gène Gene
No. accession S. cerevisiae S. cerevisiae accession No.
No. accession Yarrowia lipolytica Yarrowia lipolytica accession No.
Fonction (ou source pour 3 gènes) Function (or source for 3 genes)

TABLE 2
Genes involved in the metabolism of the peroxisomes in yeasts, particularly in
Yarrowia .lipolytica. The sequences are accessible by their names or accession
numbers at the address: http://cbi.labri.fr/Genolevures/index.php#.

| | N° accession | N° accession | |
|---|---|---|---|
| Gène | S. cerevisiae | Y. lipolytica | Fonction (ou source pour 3 gènes) |
| PEX1 | YKL197c | YALI0C15356g | AAA-peroxin |
| PEX2 | YJL210W | YALI0F01012g | RING-finger peroxin which functions in |
| | | | peroxisomal matrix protein import |
| PEX3 | YDR329c | YALI0F22539g | Peroxisomal membrane protein (PMP) |
| PEX4 | YGR133w | YALI0E04620g | Peroxisomal ubiquitin conjugating |
| | | | enzyme |
| PEX5 | YDR244w | YALI0F28457g | Peroxisomal membrane signal receptor |
| PEX6 | YNL329c | YALI0C18689g | AAA-peroxin |
| PEX7 | YDR142c | YALI0F18480g | Peroxisomal signal receptor |
| PEX8 | YGR077c | / | Intraperoxisomal organizer of the |
| | | | peroxisomal import machinery |
| PEX9 | / | YALI0E14729g | Peroxisomal integral membrane protein |
| PEX10 | YDR265w | YALI0C01023g | Peroxisomal membrane E3 ubiquitin |
| | | | ligase |
| PEX11 | YOL147c | YALI0C04092g | Peroxisomal membrane protein |
| PEX12 | YMR026c | YALI0D26642g | C3HC4-type RING-finger peroxisomal |
| | | | membrane peroxin |
| PEX13 | YLR191w | YALI0C05775g | Integral peroxisomal membrane |
| PEX14 | YGL153w | YALI0E9405g | Peroxisomal membrane peroxin |
| PEX15 | YOL044w | / | Phosphorylated tail-anchored type II |
| | | | integral peroxisomal membrane protein |
| PEX16 | / | YALI0E16599g | Intraperoxisomal peripheral membrane |
| | | | peroxin |
| PEX17 | YNL214w | / | Peroxisomal membrane peroxin |
| PEX18 | YHR160c | / | Peroxin |
| PEX19 | YDL065c | YALI0B22660g | Chaperone and import receptor |
| PEX20 | / | YALI0E06831g | Peroxin |
| PEX21 | YGR239c | / | Peroxin |
| PEX22 | YAL055w | / | Putative peroxisomal membrane protein |
| PEX23 | PEX30 | YALI0D27302g | Integral peroxisomal membrane peroxin |
| | (YLR324w) | | |
| | PEX31 | | |
| | (YGR004w) | | |
| | PEX32 | | |
| | (YBR168w) | | |
| PEX25 | YPL112c | YALI0D05005g | Peripheral peroxisomal membrane |
| | | | peroxin |
| PEX27 | YOR193w | / | Peripheral peroxisomal membrane |
| | | | protein |
| PEX28 | YHR150w | YALI0D11858g | Peroxisomal integral membrane peroxin |
| | | YALI0F19580g | |
| PEX29 | YDR479c | YALI0F19580g | Peroxisomal integral membrane peroxin |
| PEX30 | YLR324W | YALI0D27302g | Peroxisomal integral membrane protein |
| PEX31 | YGR004W | YALI0D27302g | Peroxisomal integral membrane protein |
| PEX32 | YBR168w | YALI0D27302g | Peroxisomal integral membrane protein |

Key to Table 2:
Gène Gene
No. accession S. cerevisiae S. cerevisiae accession No.
No. accession Yarrowia lipolytica Yarrowia lipolytica accession No.
Fonction (ou source pour 3 gènes) Function (or source for 3 genes)

TABLE 3
Strains of E. coli and Yarrowia lipolytica used.

| Strains (host | | Source or |
|---|---|---|
| strain) | Genotype or plasmid | reference |
| E. coli | | |
| strains | | |
| Mach1T1 | ΔrecA1398 endA1 tonA φ80ΔlacM15 ΔlacX74 hsdR(r_{K}m_{K}⁺) | Invitrogen |
| JME1128 | JMP 62 URA3 Ex-pTEF-GPD1 | This work |
| Y. lipolytica | | |
| strains | | |
| W29 | MATa WT | Aggelis et |
| | | Sourdis, 1997 |
| PO1d | MATa ura3-302 leu2-270 xpr2-322 | Barth et |
| | | Gaillardin, |
| | | 1996 |
| JMY1202 | MATa ura3-302 leu2-270 xpr2-322 Δgut2::URA3 | Beopoulos |
| | | et al., |
| JMY1233 | MATa ura3-302 xpr2-322 Δleu2 Δpox1-6 | Beopoulos |
| | | et al., |
| JMY1346 | MATa ura3-302 leu2-270 xpr2-322 Δgut2 | Beopoulos |
| | | et al., |
| JMY1351 | MATa ura3-302 xpr2-322 Δleu2 Δpox1-6 Δgut2::URA3 | Beopoulos |
| | | et al., |
| JMY1367 | MATa ura3-302 xpr2-322 Δleu2 Δpox1-6 Δgut2 | Beopoulos |
| | | et al., |
| JMY1748 | MATa ura3-302 leu2-270 xpr2-322 + pTEF-GPD1-URA3 | This work |
| JMY1789 | MATa ura3-302 xpr2-322 Δleu2 Δpox1-6 Δgut2 + pTEF- | This work |
| | GPD1-URA3 | |
| JMY1796 | MATa ura3-302 leu2-270 xpr2-322 Δgut2 + pTEF-GPD1- | This work |
| | URA3 | |
| JMY1972 | MATa ura3-302 xpr2-322 Δleu2 Δpox1-6 + pTEF-GPD1- | This work |
| | URA3 | |

TABLE 4
Primers used for constructing the vectors and for the Q-PCR. For each primer
pair, the first one is the “forward” primer and the second one the
“reverse” primer.

| Vecteur | Gène | Amorces pour construction de vecteur |
|---|---|---|
| JME1128 | GPD1 | SEQ ID NO: 3 |
| | | GCGGATCCCACAATGAGCGCTCTACTTCGATCGTCCC |
| | | SEQ ID NO: 4 |
| | | GCGCCTAGGCTAGTTGGCGTGGTAAAGAATCTCGGG |
| Gènes | N^{o} Accession | Amorces pour analyses de Q-PCR |
| ACT | | SEQ ID NO: 5 |
| | | TCCAGGCCGTCCTCTCCC |
| | | SEQ ID NO: 6 |
| | | GGCCAGCCATATCGAGTCGCA |
| ARE1 | YALI0F06578g | SEQ ID NO: 7 |
| | | TCCTCAAGCGACACGTCTA |
| | | SEQ ID NO: 8 |
| | | CAGCAACAGCAGGTATCC |
| ARE2 | YALI0D07986g | SEQ ID NO: 9 |
| | | TTCTCATCTTCCAGTACGCCTA |
| | | SEQ ID NO: 10 |
| | | GGCAATAAGATTGAGACCGTT |
| DGA1 | YALI0E32769g | SEQ ID NO: 11 |
| | | TGTACCGATTCCAGCAGT |
| | | SEQ ID NO: 12 |
| | | GGTGTGGGAGATAAGGCAA |
| GPD1 | YALI0B02948g | SEQ ID NO: 13 |
| | | CGAGACTACCGTTGCTTAC |
| | | SEQ ID NO: 14 |
| | | CAACAACGTTCTTAAGGGC |
| GUT2 | YALI0F00484g | SEQ ID NO: 15 |
| | | TGTGTGGAACCTCGACTACAA |
| | | SEQ ID NO: 16 |
| | | CATCCAGAAGTAGGGAAGC |
| LRO1 | YALI0E16797g | SEQ ID NO: 17 |
| | | CTCCGCCGACTTCTTTATG |
| | | SEQ ID NO: 18 |
| | | GAAGTATCCGTCTCGGTG |
| SCT1 | YALI0C00209g | SEQ ID NO: 19 |
| | | GAGATTGTGTCCGACACT |
| | | SEQ ID NO: 20 |
| | | TTTGTCGAATACGGATCGGT |
| TGL3 | YALI0D17534g | SEQ ID NO: 21 |
| | | CAAGATTGTCTCTCCCTACG |
| | | SEQ ID NO: 22 |
| | | GTACTCGGATCAGGTTCAC |
| TGL4 | YALI0F10010g | SEQ ID NO: 23 |
| | | GTTCGACAAGGAGCCTATT |
| | | SEQ ID NO: 24 |
| | | GGTCAGATGCGGATGATAAAG |

Key to Table 4
Vecteur Vector
Gène(s) Gene(s)
Amorces pour construction de vecteur Primers for vector construction
No. Accession Accession No.
Amorces pour analyses de Q-PCR Primers for Q-PCR analyses

TABLE 5
Fatty acid profiles for the wild and mutant strains. The strains were cultivated
in YNB with 0.5% glucose and 3% oleic acid (70% pure). The values indicated
correspond to the average of three different experiments after 24 hours of
cultivation. The standard deviation was <10% of the values indicated.

| | | | | | | | | Δgut2 |
|---|---|---|---|---|---|---|---|---|
| | | PO1d | | Δgut2 | | Δpox1-6 | Δgut2 | Δpox1-6 |
| | PO1d | GPD1+ | Δgut2 | GPD1+ | Δpox1-6 | GPD1+ | Δpox1-6 | GPD1+ |
| C16:0 | 6.0 | 5.1 | 5.0 | 4.9 | 4.0 | 4.1 | 2.6 | 5.3 |
| C16 1(n-9) | 1.1 | 0.9 | 13.7 | 14.4 | 0.3 | 0.4 | 0.2 | 0.4 |
| C16:1(n-7) | 4.3 | 6.0 | 6.5 | 6.5 | 3.8 | 5.6 | 3.2 | 8.1 |
| C18:0 | 0.6 | 0.5 | 0.3 | 0.5 | 0.7 | 0.6 | 0.7 | 0.8 |
| C18:1(n-9) | 57.5 | 56.9 | 48.1 | 46.1 | 68.1 | 62.8 | 60.1 | 59.7 |
| C18:2(n-6) | 24.8 | 21.4 | 22.0 | 22.2 | 14.1 | 18.3 | 16.2 | 15.8 |
| Total | 94.3 | 90.9 | 95.8 | 94.6 | 91.0 | 91.8 | 83.0 | 90.1 |

TABLE 6
Profile of the metabolites produced by NMR analysis. The metabolites were identified
and quantified over a series of experiments, by using maleate and methyl-phosphonate
as internal standards. Two separate experiments were carried out using two separate cultures
and yielded similar results. A representative result is presented. G3P, glycerol-3-phosphate,
GPC, glycerol phosphorylcholine, GPE, glycerol phosphorylethanolamine.

| Metabolites^{a} | | | | | | | |
|---|---|---|---|---|---|---|---|
| | | | | | | | Δgut2 |
| | PO1d | | Δgu12 | | Δpox1-6 | Δgut2 | Δpox1-6 |
| PO1d | GPD1⁺ | Δgut2 | GPD1⁺ | Δpox1-6 | GPD1⁺ | Δpox1-6 | GPD1⁺ |

¹³C metabolites (mmoles/g of dry mass)

| | | | | | | | | |
|---|---|---|---|---|---|---|---|---|
| Glycerol | 8.5 | 4.2 | 5.2 | 2 | 4.4 | 3 | 0.8 | 1.5 |
| Malate | 17.6 | 26.1 | 16.8 | 8 | 7.1 | 2.5 | 4 | 2.2 |
| Citrate | 2 | 1.2 | 1.3 | 0.8 | 0.7 | 0.5 | 0.5 | 0.7 |
| Succinate | 15.2 | 17.6 | 9.6 | 7.8 | 7.4 | 4.1 | 3.2 | 2.7 |

³¹P metabolites (μmoles/g of dry mass)

| | | | | | | | | |
|---|---|---|---|---|---|---|---|---|
| G3P | 5 | 7.7 | 14.4 | 28.5 | 7 | 10.3 | 14.6 | 11.7 |
| GPC | 67 | 50 | 80 | 112 | 69 | 66 | 75 | 52 |
| GPE | 10 | 7 | 11.1 | 37.4 | 7.7 | 6.4 | 10.8 | 7.8 |

1.-26. (canceled)

27. An oleaginous strain of yeast in which the beta-oxidation of fatty acids is deficient and which overexpresses the GPD1 gene, said mutant strain being capable of accumulating lipids.

28. The oleaginous strain of yeast of claim 27, wherein the yeast is of a genera selected from the group consisting of Candida, Cryptoccocus, Rhodotorula, Rhizopus, Trichosporon, Lypomyces and Yarrowia.

29. The oleaginous strain of yeast of claim 27, wherein the yeast is Yarrowia lipolytica, Rhodotura glutinis or Rhodosporidium torulides.

30. The oleaginous strain of yeast of claim 27, wherein the yeast is Yarrowia lipolytica.

31. The oleaginous strain of yeast of claim 27, which comprises at least one loss-of-function mutation in at least one gene selected from the group consisting of the PEX genes, the POX genes, the MFE1 gene and the POT1 gene.

32. The oleaginous strain of yeast of claim 27, which comprises at least one loss-of-function mutation in at least one of the genes selected from the group consisting of POX1, POX2, POX3, POX4, POX5 and POX6.

33. The oleaginous strain of yeast of claim 27, which comprises a loss-of-function mutation in each of the genes POX1, POX2, POX3, POX4, POX5 and POX6.

34. The oleaginous strain of yeast of claim 27, which further comprises at least one additional mutation in at least one gene encoding an enzyme involved in the metabolism of the fatty acids.

35. The oleaginous strain of yeast of claim 34, wherein the additional mutation has the effect of further increasing the capacity of the strain to accumulate lipids.

36. The oleaginous strain of yeast of claim 35, which is deficient in each of the products of the TGL3 and TGL4 genes.

37. The oleaginous strain of yeast of claim 35, which comprises an inactivated GUT2 gene.

38. The oleaginous strain of yeast of claim 34, wherein the additional mutation has the effect of altering the profile of stored fatty acids.

39. The oleaginous strain of yeast of claim 38, which comprises an inactivated YALIOB10153g gene.

40. The oleaginous strain of yeast according to claim 27, which expresses a gene encoding an enzyme selected from the group consisting of a Δ8 desaturase, a Δ12 desaturase and a Δ15 desaturase.

41. The oleaginous strain of yeast of claim 40, wherein said gene encodes a Δ12 desaturase.

42. The oleaginous strain of yeast of claim 41, wherein said gene is the YALIOB10153g gene of Yarrowia lipolytica.

43. A method for obtaining an oleaginous strain of yeast according to claim 27, comprising:
(a) inactivating at least one gene of interest controlling beta-oxidation in the oleaginous strain of yeast, and
(b) transforming the oleaginous strain of yeast by means of a polynucleotide enabling expression of the gene GPD1.

44. The method according to claim 43, wherein the oleaginous strain of yeast is a strain of Yarrowia lipolytica.

45. The method according to claim 43, wherein step (a) includes:
i) selecting a gene of interest controlling beta-oxidation that one wishes to delete,
ii) constructing a disruption cassette by PCR (“Polymerase Chain Reaction”) or by cloning,
iii) introducing a selection marker containing recombination sequences on both sides enabling recombination in order to eliminate the marker,
iv) selecting the strains with the gene of interest deleted and verifying the deletion,
v) transforming using a vector that enables expression of the recombinase, and
vi) isolating a clone having the gene of interest deleted and having lost the recombinase expression plasmid.

46. The method according to claim 45, wherein the recombination sequences are loxP, loxR or both and wherein the recombinase is the Cre recombinase.

47. The method according to claim 43, wherein step (a) is repeated to inactivate one or more additional genes.

48. The method according to claim 43, characterized in that the GPD1 gene of step (b) is under the control of a promoter sequence and a terminator sequence, wherein the promoter sequence is selected from the group consisting of the POX2 promoter, the LIP2 promoter, the FBA promoter, the GPM promoter, the YAT1 promoter, the GPAT promoter, the TEF promoter, the hp4d hybrid promoter and XPR2 hybrid promoters and wherein the terminator sequence is selected from the group consisting of the terminator sequence of the PGK1 gene and the terminator sequence of the LIP2 gene.

49. A mutant oleaginous strain of yeast obtained by the method of claim 43 in which the beta-oxidation of fatty acids is deficient, which overexpresses the GPD1 gene, and which is capable of accumulating lipids.

50. A lipid synthesis method comprising:
(1) cultivating an oleaginous strain of yeast of claim 27 in an appropriate medium, and
(2) harvesting the lipids produced during cultivation.

51. The method of claim 50 wherein the lipids are fatty acids or triglycerides.

52. The method of claim 50 wherein the oleaginous strain of yeast is of a genera selected from the group consisting of Candida, Cryptoccocus, Rhodotorula, Rhizopus, Trichosporon, Lypomyces and Yarrowia.

53. The method of claim 50 wherein the oleaginous strain of yeast is Yarrowia lipolytica, Rhodotura glutinis or Rhodosporidium torulides.

54. The method of claim 50 wherein the oleaginous strain of yeast is Yarrowia lipolytica.
